(19) United States
(12) Patent Application Publication
(10) Pub. No.: US 2021/0369187 A1
(43) Pub. Date: Dec. 2, 2021

(54) NON-CONTACT CHEWING SENSOR AND PORTION ESTIMATOR

(71) Applicant: THE BOARD OF TRUSTEES OF THE UNIVERSITY OF ALABAMA, Tuscaloosa, AL (US)

(72) Inventors: Viprav Bhaskar Raju, Tuscaloosa, AL (US); Edward Sazonov, Northport, AL (US)

(21) Appl. No.: 17/211,097

(22) Filed: Mar. 24, 2021

Related U.S. Application Data
(60) Provisional application No. 63/030,701, filed on May 27, 2020.

(51) Int. Cl. A61B 5/00; A61B 5/11

(52) U.S. Cl. CPC A61B 5/4542; A61B 5/0077; A61B 2562/0238; A61B 5/6803; A61B 2562/0219; A61B 5/1113

(57) ABSTRACT

In an embodiment, a non-contact chewing sensor is provided. The chewing sensor includes an optical proximity sensor specifically designed to monitor chewing. An IR emitter/receiver pair of the chewing sensor may be positioned over a muscle of a user that is involved in the chewing process such as the temporalis muscle. The IR emitter of the sensor emits IR light onto the surface of the skin covering the muscle where it is reflected. When the user chews, the amount of light that is received by the IR receiver changes due to the activation of the muscle. The amount of light received can be used as a signal to determine when the user is chewing and likely eating.

### CROSS-REFERENCE TO RELATED APPLICATIONS

This application claims priority to U.S. Provisional Patent Application Ser. No. 63/030,701 filed on May 27, 2020 entitled NON-CONTACT CHEWING SENSOR AND PORTION ESTIMATOR. The disclosure of which is hereby incorporated by reference in its entirely.

### STATEMENT OF GOVERNMENT SUPPORT

This invention was made with government support under grant No. 25165 awarded by the National Institutes of Health. The government has certain rights in the invention.

### BACKGROUND

Obesity is a growing global epidemic and are major contributors to the development of chronic diseases such as type 2 diabetes, asthma, cardiovascular diseases, cancers, and musculoskeletal disorders. Obesity is often considered to be a result of a sustained energy imbalance whereby energy intake (El) exceeds energy expenditure (EE), causing an accumulation of fat in the body. An accurate understanding of El can help develop strategies for weight loss interventions. Despite decades of obesity research, an accurate estimation of El in human is still a difficult challenge.

Energy intake is directly related to eating behavior, which is a complex interaction of a variety of physiological, emotional, social, cultural, environmental, and economic factors that influence the timing of an eating episode, amount of food intake, food choice and/or selection, and the way in which food is consumed. To address issues with accurate measurement of energy intake and eating behavior, research involving the automatic detection of eating episodes, recognition of the foods being consumed and measurement of the quantity and the manner of consumption in an eating episode is rapidly progressing.

### SUMMARY

In an embodiment, a non-contact chewing sensor is provided. The chewing sensor includes an optical proximity sensor specifically designed to monitor chewing. The chewing sensor may be attached to eyeglasses of a user or incorporated into an augmented reality headset of the user. The chewing sensor may include IR light emitter and IR light receiver. The IR emitter/receiver pair of the chewing sensor may be positioned over a muscle of the user that is involved in the chewing process such as the temporalis muscle. The IR emitter of the sensor emits IR light onto the surface of the skin covering the muscle where it is reflected. When the user chews, the amount of light that is received by the IR receiver changes due to the activation of the muscle. The amount of light received can be used as a signal to determine when the user is chewing and likely eating. Determining when and how long a user is eating may be useful for a variety of applications such as weight loss and scientific research. The chewing sensor may further include an eye gaze-aligned camera and other sensors that may be used to estimate portion sizes or amounts of foods that have been eaten.

In an embodiment, a food type and portion estimation sensor is provided. The portion estimation sensor comprises: a housing; a processing component contained in the housing; a distance sensor contained in the housing; a camera contained in the housing; and an inertial measurement unit contained in the housing. The processing component may be adapted to: receive an image of food from the camera; receive data measured by the inertial measurement unit; based on the data measured by the inertial measurement unit, determine a viewing angle of the camera; receive a distance measurement from the distance sensor; and based on the determined viewing angle and the received distance, estimate a size of the food in the image; based on the image captured by the camera, recognize the food type being consumed.

In an embodiment, a portion estimation sensor is provided. The sensor includes a housing, a processing component contained in the housing, a distance sensor contained in the housing; a camera contained in the housing; and an inertial measurement unit contained in the housing. The processing component is adapted to: receive an image of a vessel from the camera; receive data measured by the inertial measurement unit; based on the data measured by the inertial measurement unit, determine a viewing angle of the camera; receive a distance measurement from the distance sensor; and based on the determined viewing angle and the received distance, estimate a size of the vessel in the image.

Implementation may include some or all of the following features. The data measured by the inertial measurement unit may include heading, pitch, and roll angle. Estimating a size of the vessel may include: calculating a first plane of an eating surface that includes the vessel; calculating a height of the vessel; calculating a second plane that includes a top of the vessel based on the calculated height; and estimating the size of the vessel based on the first plane, the second plane, the height, and the image. The sensor may further, based on the estimated size of the plate, estimate a size of food in the vessel. The housing may be adapted to attach to a pair of glasses. The housing may be part of an AR or VR headset or goggles.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is an illustration of an example chewing sensor;

FIG. 2 is an illustration of an example user with an indication of where the IR emitter may face when detecting chewing;

FIG. 3 is another illustration of an example chewing sensor;

FIG. 4 is an illustration of an example graph showing analogue-to-digital values corresponding to times associated with chewing and not chewing;

FIG. 5 are illustrations of a geometry that may be used to determine a size of a plate in front of the wearer of the sensor;

FIG. 6 is an illustration of an example chewing sensor with camera, IMU, and distance sensor;

FIG. 7 is an illustration of an example chewing sensor being used to determine a height of a bowl;

FIG. 8 is an illustration of a method for estimating a food portion by a wearable sensor;

FIG. 9 is an illustration of a method for detecting when a wearer of a sensor is chewing; and

FIG. 10 is an illustration of an exemplary computing environment in which example embodiments and aspects may be implemented.

### DETAILED DESCRIPTION

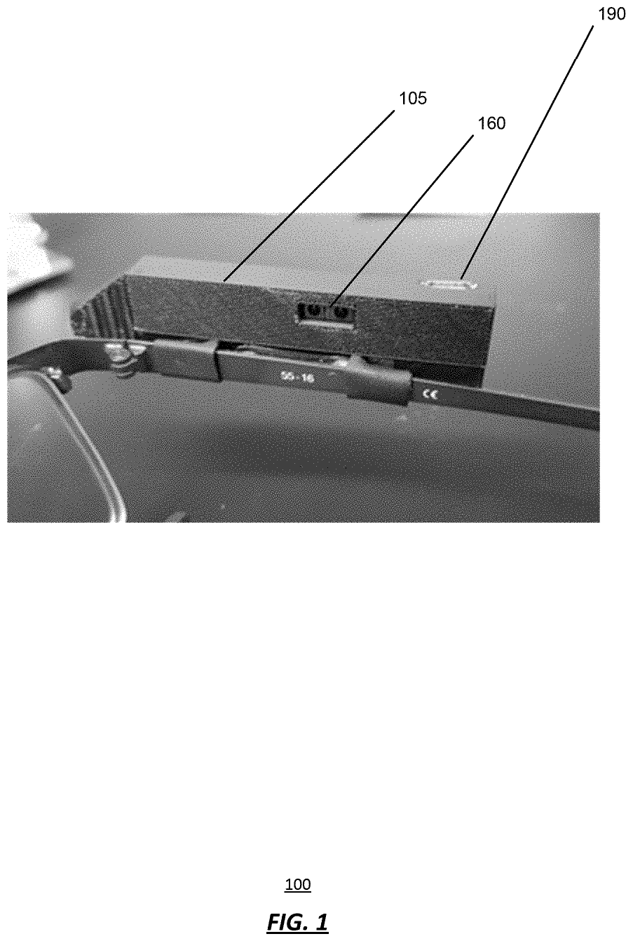

FIG. 1 is an illustration of an example chewing sensor 100. As shown, the chewing sensor 100 includes components such as a housing 105, an infra-red (“IR”) sensor 160, and an I/O interface 190. More or fewer components may be supported.

Depending on the embodiment, the chewing sensor 100 may be configured to detect when the user or wearer is chewing (and not chewing) solid foods and/or sucking liquids. The chewing sensor 100 may further be configured to count the total number of chews or sucks made by the wearer. As may be appreciated, the number of chews made by a user may be useful for purposes of estimating the total amount of food consumed by the user over a particular time period, or for estimating the energy intake of the user.

The housing 105 of the chewing sensor 100 may be constructed from a lightweight material such as plastic. Other materials may be used. As shown, the housing 105 may be constructed to allow the chewing sensor 100 to be easily attached to the temples of a pair of eyeglasses. Depending on the embodiment, the housing 105 could be part of an existing pair of augmented reality or virtual reality glasses or headset. Furthermore, instead of glasses, the housing 105 could be adapted to be attached to a hat, helmet or other type of head-gear that can be worn by a user.

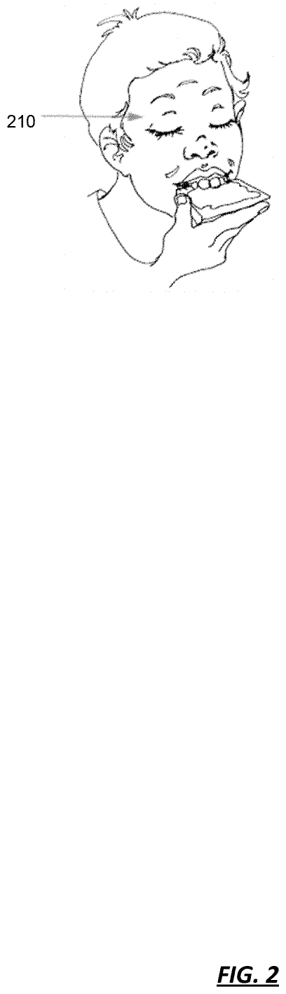

The IR sensor 160 may include an IR emitter and an IR receiver. The IR sensor 160 may be placed in the housing 105 such that when the chewing sensor 100 is worn by a user, the IR emitter is facing a muscle that is used by the user when chewing. A suitable muscle is the temporalis muscle. The temporalis muscle may be preferable because it is pronounced when chewing and less pronounced when performing other jaw moving activities such as talking. However, other muscles may be used. For example, FIG. 2 includes an arrow 210 that indicates a location of the muscle that IR emitter may face when the chewing sensor is worn by a user.

Returning to FIG. 1, the IR sensor 160 may emit IR light towards the temporalis muscle of the user continuously or in pulses. Depending on the activation/relaxation of the temporalis muscle, some of the emitted IR light may be reflected off of the skin of the user and away from the IR receiver or toward the receiver. The IR sensor 160 may interpret these changes in received IR light as chewing.

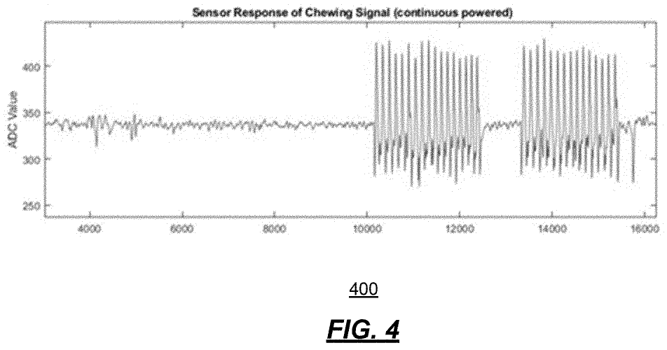

For example, FIG. 4 is an illustration of an example graph 400 showing on the y-axis the IR values received by an analog-to digital converter (ADC) connected to the IR receiver, and on the x-axis time. From 0-10000, the wearer if the chewing sensor 100 is not chewing and the value of the ADC remains fairly constant at around 350. From 10000 to approximately 12500 the wearer has begun to chew, and the value of the ADC begins to jump between 250 and 400 as the user chews. Once the wearer stops chewing at 12500 the AOC values return to 350. At 13500, the wear has begun chewing again, causing another spike in AOC value.

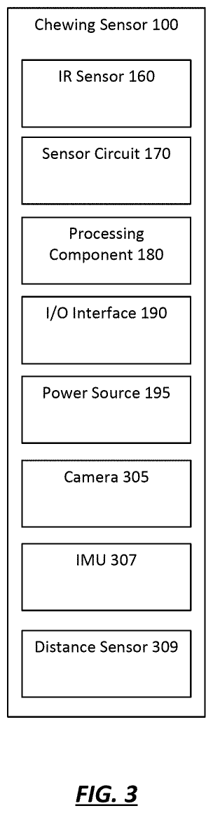

FIG. 3 is another illustration of an example chewing sensor 100. As shown, the chewing sensor 100 includes components such as an IR sensor 160, a sensor circuit 170, a processing component 180, an I/O interface 190, a power source 195, a camera 305, an IMU 307, and a distance sensor 309. More or fewer components may be supported.

The IR sensor 160 may include an IR emitter and an IR receiver as described above with respect to FIG. 1. The IR emitter may emit IR light towards a muscle such as the temporalis muscle. The IR emitter and IR receiver may be arranged such that the IR receiver receives more IR light from the IR emitter when a wearer of the chewing sensor 100 is chewing than when the wearer is not chewing.

The sensor circuit 170 may receive as an input a signal from the IR receiver, and may output another signal that may be used by the processing component 180 to determine if the wearer of the chewing sensor 100 is chewing or not chewing. The sensor circuit 170 may allow the chewing sensor 100 to use less power and operate in direct sunlight without saturating the IR receiver.

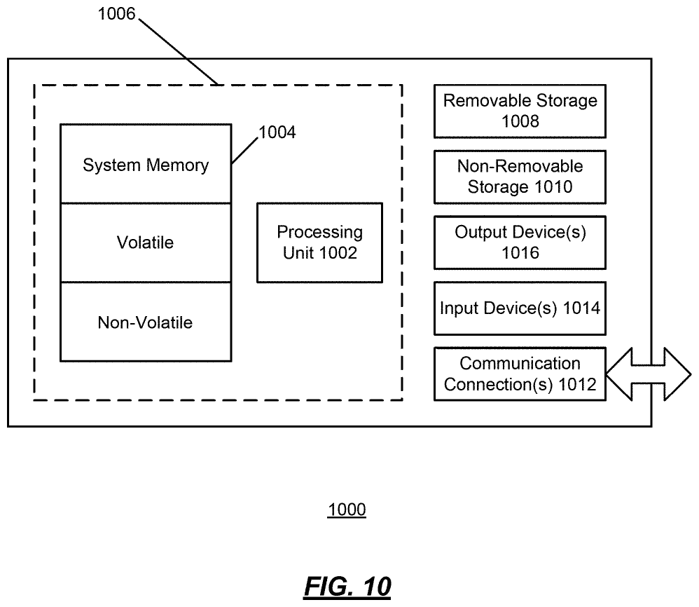

The processing component 180 may receive output from the sensor circuit 170, and based on a value of the output, may determine whether or not the wearer is chewing. Depending on the embodiment, the processing component 180 may determine that the wearer is chewing when computed metrics satisfies one or more criteria or when the output exceeds a threshold, for example. The processing component 180 may be implemented using a general-purpose computing device such as the computing device 1000 described with respect to FIG. 10.

The processing component 180 may further store data about the wearer of the chewing sensor 100. The stored data may include number of chews detected, times when each chew was detected, and a duration of each chew. Depending on the embodiment, the processing component 180 may use the data to estimate how long the wearer ate, as well as the mass or amount of food that the wearer ate.

The I/O interface 190 may allow the chewing sensor 100 to receive and transmit data. The I/O interface 190 may include a wired interface such as USB and/or a wireless interface such as Bluetooth or Wi-Fi. Other interfaces may be used. The processing component 180 may periodically provide the stored and collected data about the wearer through the I/O interface 190. For example, a health or weight loss application may receive data for a wearer of the chewing sensor 100 that may be used by the application to determine if the wearer is adhering to a weight loss or eating plan.

The power source 195 may provide power to the chewing sensor 100. An example power source 195 is a battery. Other power sources 195 may be used. Depending on the embodiment, the power source 195 may be a rechargeable battery and may receive power from an external power supply.

In some embodiments, the chewing sensor 100 described above may be modified to allow for the estimation of portion size or to improve the estimation of the amount of food ingested. In particular, the chewing sensor 100 may be modified to include the camera 305, the inertial measurement unit 307 (IMU), and the distance sensor 309.

In some embodiments, the camera 305, IMU 307, and distance sensor 309 may be mounted on or embedded into an eyeglasses frame, may be mounted on or behind an ear, or may be mounted on a headset as shown in FIG. 1. Food intake may be detected by one of the sensors described above (i.e., IR sensor). The camera 305 may be triggered by the food intake sensor, foods and beverage in the image may then be recognized using a variety of methods.

The IMU 307 may be a simple 3D accelerometer, a 6D accelerometer, or a 9D accelerometer. Other types of accelerometers, gyroscopes, or magnetometers may be used. The IMU may respond to the Earth's gravity (and magnetic field, if implemented) field and may register the head pose of the wearer in terms of heading, pitch, and roll angles (μ, φ, ψ).

Food being eaten is usually located in a plane normal (90²) to the Earth gravity field, so that the food is stationary and does not move. The food is also normally located in front of the person consuming it. Thus, the viewing angle of the camera can be estimated from simple geometric equations. For example, the viewing angle with respect to the food plane may be estimated from the pitch angle as: μ̆=90−μ. Thus, the viewing angle of the camera needed for portion size estimation may be computed from the angles measured by the IMU 307.

The distance sensor 309 (also referred to as ranging sensor or time of flight sensor) measures the distance to the scene with a high accuracy (for example, 1 mm). The optical axis of the distance sensor may be co-aligned with the optical axis of the camera 305, pointing to the known region within camera's 305 field of view. Thus, when the food being eaten is recognized to be located within this region of the image, the distance sensor 309 returns a valid distance measurement d to the food being eaten. The measured distance and view angle may then be utilized in conjunction with computer vision methods to estimate the portion size of the food being eaten. The measured portion size may be used in estimation of energy intake or combined with other metrics (e.g., the amount of food eaten as determined by the chewing sensor 100).

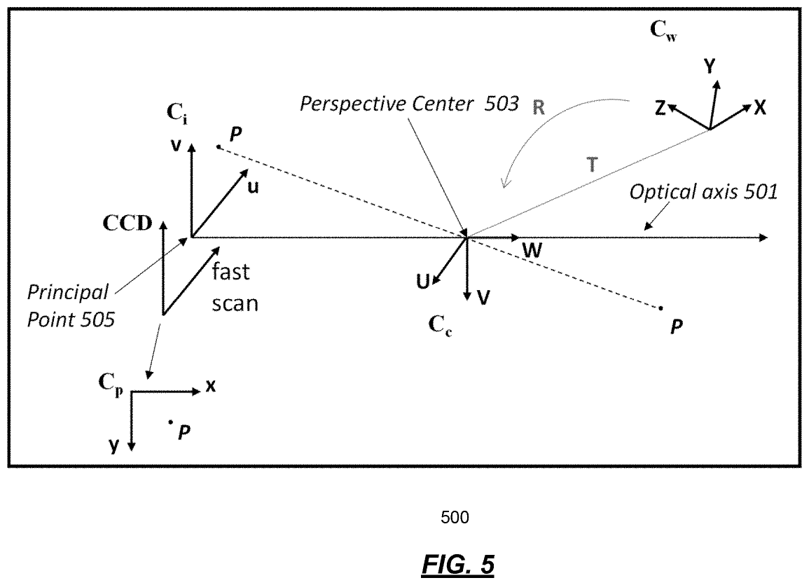

In some embodiments, the chewing sensor 100 may first determine the size of a plate in front of a wearer using a geometry 500 as shown in FIG. 5. The objective of the chewing sensor 100 is to project the points in an image in the real world coordinates. Referring to FIG. 5, let P be a point in the world, C_{w} a world coordinate system and (X Y Z)^{t} be the coordinates of P in C_{c}. A camera coordinate system, C_{c}, may be defined to have its W-axis parallel with the optical axis 501 of a lens of the camera 305, its U-axis parallel with the u-axis of Cᵢ (image co-ordinate plane) and origin located at a perspective center 503. Let (U V W)^{t} be the coordinates of P in C_{c}. The coordinates (U V W)^{t} are related to (X Y Z)^{t} by a rigid body coordinate transformation and described in the following equation 1 where R is a 3×3 rotation matrix and T is a 3×1 translation vector:

$$( \begin{matrix}U \\ V \\ W\end{matrix} ) = R ( \begin{matrix}X \\ Y \\ Z\end{matrix} ) + T \qquad ( 1 )$$

The principal point 505 is the intersection of the imaging plane with the optical axis 501. The 2D image coordinate system, Cᵢ, may be defined to be in the image plane with its origin located at the principal point 505, u-axis in the fast scan direction and v-axis in the slow scan direction of a camera sensor. Let p be the projection of P onto the image plane and let (ū ū)^{t} be the coordinates of p in Cᵢ. Then (ū ū)^{t} are given by the following equation 2:

$$( \begin{matrix}\overline{u} \\ \overline{υ}\end{matrix} ) = \frac{{f}_{c}}{W} ( \begin{matrix}U \\ V\end{matrix} ) \qquad ( 2 )$$

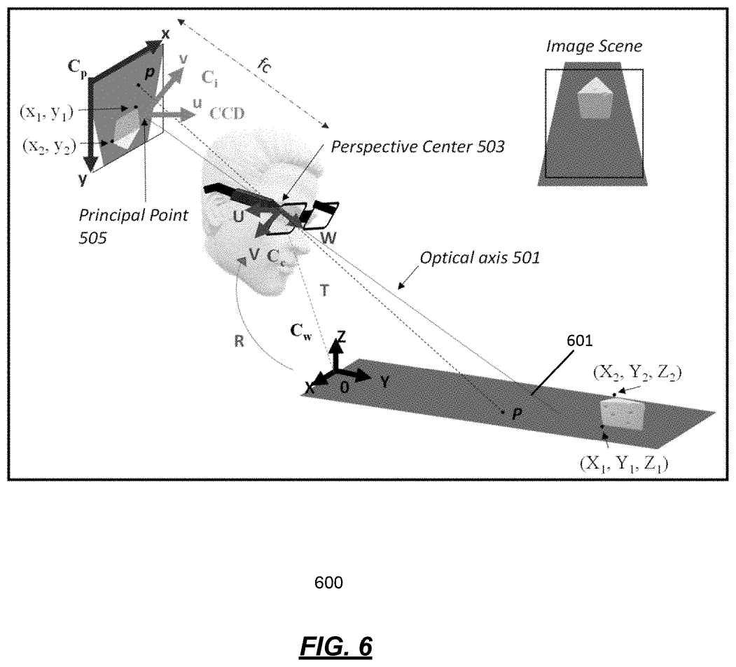

Referring to FIG. 6, radial lens distortion of the camera 305 may be incorporated into the model in the following way. Let (u v)^{t} be the actual observed image point after being subject to lens distortion. Then (u v)^{t} is related to (ū ū)^{t} by the following equation 3 where K_{c} is a coefficient which controls the amount of radial distortion:

$$( \begin{matrix}u \\ v\end{matrix} ) = ( \begin{matrix}\overline{u} \\ \overline{υ}\end{matrix} ) [ 1 + {K}_{c} ( {\overline{u}}^{2} + {\overline{υ}}^{2} ) ] \qquad ( 3 )$$

Finally, the image sampling performed by a camera sensor (CCD) of the camera 305 may be modeled. Let Cₚ be the pixel coordinate system associated with the digital image. The pixel coordinates are related to the image coordinates by the following equation 4 where s_{c}^{x}; s_{c}^{y} are scale factors (pixel/mm), c_{c}^{x}; c_{c}^{y} are the pixel coordinates of the principal point and K_{c} is the distortion coefficient (pixel/mm):

$$( \begin{matrix}x \\ y\end{matrix} ) = ( \begin{matrix}{s}_{c}^{x} & {K}_{c} \\ 0 & {s}_{c}^{y}\end{matrix} ) ( \begin{matrix}u \\ v\end{matrix} ) + ( \begin{matrix}{c}_{c}^{x} \\ {c}_{c}^{y}\end{matrix} ) \qquad ( 4 )$$

Equation 5 is given by:

$$( \begin{matrix}x \\ y\end{matrix} ) = f ( XYZ ) \qquad ( 5 )$$

For purposes of volume estimation, the inverse of the equation 5 is given by the following equation 6:

$$( \begin{matrix}X \\ Y \\ Z\end{matrix} ) = {f}^{- 1} ( x y ) \qquad ( 6 )$$

With the known orientation provided by the IMU 307, a right angle between the surface of the lens and the optical axis 501, and the projection relationship in equation 5, it can be shown that the inverse of function ƒ in equation 6 exists for a tabletop 601 according to the following equation 7:

$$( \begin{matrix}X \\ Y \\ 0\end{matrix} ) = {f}^{- 1} ( x y ) \qquad ( 7 )$$

Note that Z=0 in equation 7 represents the plane equation of the tabletop 601.

Also, it may be assumed that the roll and yaw of the sensor is zero which results int the following equation 8:

$$R = [ \begin{matrix}1 & 0 & 0 \\ 0 & cos θ & - sin θ \\ 0 & sin θ & cos θ\end{matrix} ] \qquad ( 8 )$$

From equation 4, the world coordinates of the tabletop 601 are related to the pixel coordinates by the following equation 9:

$$( \begin{matrix}u \\ v\end{matrix} ) = ( \begin{matrix}1 / {s}_{c}^{x} & 0 \\ - K c & 1 / {s}_{c}^{y}\end{matrix} ) + ( \begin{matrix}x - {c}_{c}^{x} \\ y - {c}_{c}^{y}\end{matrix} ) \qquad ( 9 )$$

To calculate the translational and rotation matrices, the sensor pitch from the IMU 307 and distance readings from the distance sensor 309 are used. Depending on the embodiment, the camera 305 on the sensor 100 has an offset of 21 degrees.

Using the following equation 10, where dtof=distance the distance between the sensor 100 and the eating surface as determined by the distance sensor 309, the equation 11 can be derived as:

$$\begin{matrix}h = dtof \times tan ( ω ) & ( 10 ) \\ W = \frac{h \cdot sin ( θ )}{cos ( θ ) \frac{v \cdot sin ( θ )}{{f}_{c}}} & ( 11 )\end{matrix}$$

From the equation 9 the following equation 12 may be derived:

$$( \begin{matrix}U \\ V\end{matrix} ) = \frac{W}{{f}_{c}} ( \begin{matrix}u \\ v\end{matrix} ) \qquad ( 12 )$$

Finally, the equation 13 is obtained where T=[0; −h; 0]:

$$( \begin{matrix}X \\ Y \\ 0\end{matrix} ) = {R}^{- 1} ( ( \begin{matrix}U \\ V \\ W\end{matrix} ) - T ) \qquad ( 13 )$$

Equation 13 gives us the plane of the eating surface (Z=0).

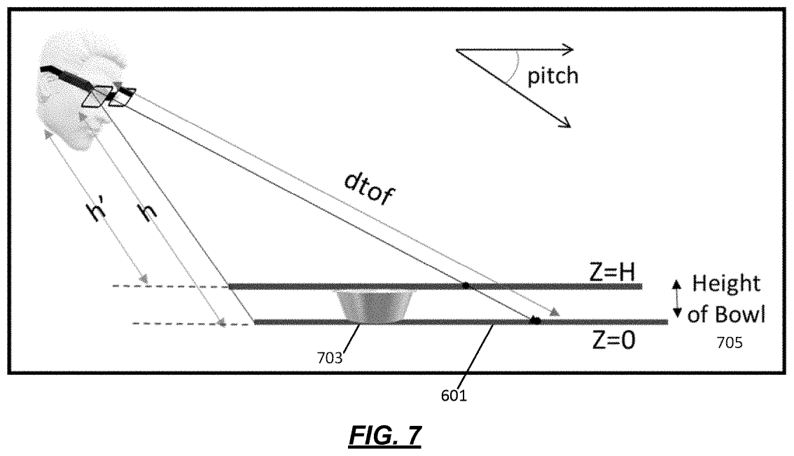

Continuing to FIG. 7, in some embodiments, after calculating the plane of the eating surface (e.g., the table 601), the processing component 180 of the sensor 100 may calculate the size and/or dimensions of a vessel 703 on the eating surface. Examples of vessels include plates, bowls, and other food containers.

First the height 705 of the vessel is measured along the y-axis as shown in FIG. 7. The height 705 is just a projection on the y-axis and the true height is calculated as below in the equation 14. Here, the assumption is that the vessel sides are flat and not curved.

H=tan(ω)×H′ (14)

Once, the height 707 of the vessel is calculated, the equation of the plane Z=H is obtained instead of Z=0. FIG. 7 depicts the changes in the parameters for obtaining the adjusted plane equation. The adjusted plane equation is given by the following equation 15 where h is calculated as in the equation 10:

h′=h−(H×sec(ω)) (15)

Once h′ is obtained, the processing component 180 may plug h′ into the equation 11 followed by equations 12 and 13.

Once the equation of the plane is determined by the processing component 180 for both the table 601 and the top of the vessel 703, the processing component 180 may use the equation of the plane to calculate the dimensions of the vessel 703. The processing component 180 may calculate the dimensions of the vessel 703 based on where the vessel 703 intersects the planes Z=0 and Z=H in the image captured by the camera 305.

As may be appreciated, once the dimensions of the vessel 703 have been calculated, the processing component 180 may use the dimensions to calculate an amount of food contained in the vessel 703 (i.e., portion size). The processing component 180 may calculate the amount of food by determining the percentage of the vessel 703 that is taken up by the food. The percentage may be determined from the image captured by the camera 305. After determining the percentage, the amount of food may be determined using the percentage and the dimensions of the vessel 703. For example, the processing component 180 may determine that based on the dimensions of the vessel 703, the vessel 703 likely holds 12 ounces. The processing component 180 may further determine from the image of the vessel 703 that the food in the bowl takes up 70% of the vessel 703. The processing component 180 may then determine that the portion of food is approximately 8.4 ounces.

Once the portion size is determined, the processing component 180 may store the portion size along with other information about a current meal such as when the wearer of the chewing sensor 100 started eating, how long they ate, and how much they ate. The information may later be retrieved or viewed by the wearer, incorporated into a fitness or weight-loss application, or may be provided to a doctor or physician associated with the wearer.

The processing component 180 may further use computer vision techniques to identify the type of food being consumed by the wearer in the vessel 703. For example, a computer vision model that is trained to identify types of food may be applied to the image generated by the camera 305 and may generate a guess or estimate of the food that is in the vessel 703. Depending on the food in the vessel 703, the processing component 180 may retrieve known caloric information associated with the food, and in combination with the determined portion size, may determine the total number of calories eaten by the wearer of the sensor 100. The determined calories may be stored by the processing component 180.

As may be appreciated, the chewing sensor 100 as described above may be used to not only collect information about the calories consumed by the wearer, but to actively monitor the calories being eaten by the wearer and to notify the wearer when they have exceeded an allotted amount of calories. For example, a wearer of the chewing sensor 100 would like to lose weight is restricted to eat no more than 500 calories at each meal. Based on information from the IR sensor 160 of the camera 305, the processing component 180 may determine that the wearer is eating, and in response, may calculate the size of the portion of the food in a vessel 703 that the wearer is eating from. The processing component 180 may then use computer vison techniques to guess the food that is being eaten by the wearer. Based on the guess, the processing component 180 may determine the total amount of calories in the vessel 703.

As the wearer eats the food, the processing component 180 may continue to receive images from the camera 305 and may continuously recalculate the amount of food that remains in the vessel 703. Based on the amount of food remaining, the processing component 180 may determine the total amount of calories that have been consumed by the wearer. Once the total amount exceeds the threshold amount (e.g., 500 calories) the sensor 100 may vibrate, beep, or take some action to indicate to the wearer they have exceeded their calorie amount for the meal.

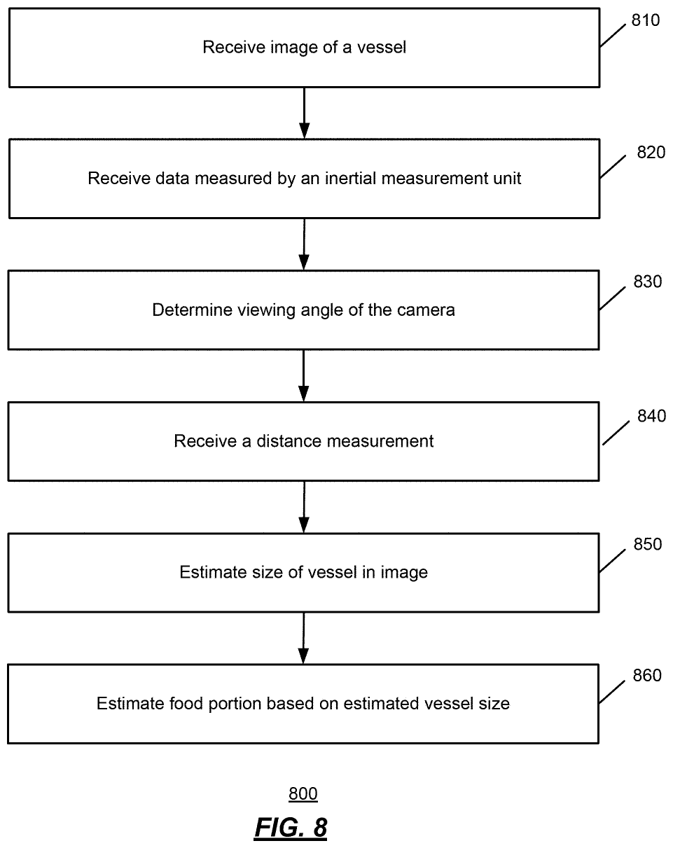

FIG. 8 is an illustration of a method 800 for estimating a food portion by a wearable sensor 100.

At 810, an image of a vessel is received. The image of the vessel may be received from a camera 305 of the sensor 100. The sensor 100 may be worn by a wearer on their head. For example, the sensor 100 may be attached to a pair of glasses or may be integrated into an AR headset. Depending on the embodiment, the image may have been taken by the camera 305 in response to the processing component 180 detecting that the wearer has begun eating. For example, the sensor 100 may detect movements of a muscle associated with eating using the IR sensor 160, or the processing component 180 may detect that the wearer has spoken a word, pressed a button, or made a gesture that is associated with eating. The vessel may be a plate, bowl, or other food container such as a takeout container.

At 820, data measured by an inertial measurement unit is received. The data may be received by the processing component 180 from the IMU 307. The data may include heading, pitch, and roll angle, for example.

At 830, a viewing angle of the camera is determined. The viewing angle may be determined by the processing component 180 using the data provided by the IMU 307.

At 840, a distance measurement is received. The distance measurement may be received by the processing component 180 from the distance sensor 309. The distance measurement may be the distance between the plate and the sensor 100 worn by the wearer.

At 850, a size of the vessel in the image is estimated. The size of the vessel may be estimated by the processing component 180 using the viewing angle and the distance measurement. In embodiments where the vessel is flat or mostly flat (e.g., plates), the processing component 180 may calculate a first plane that corresponds to an eating surface that the vessel is resting on and may estimate the size of the vessel based on the calculated first plane only.

In embodiments where the vessel 703 is a bowl or has sides, the processing component 180 may additionally estimate a height of the vessel 703 using the distance measurement and viewing angle. The processing component 180 may then calculate a second plane that includes the top of the vessel 703. The first plane may represent a bottom of the vessel. The processing component 180 may then use the first plane, the second plane, and the image to estimate the size of the vessel 703. Depending on the embodiment, estimating the size of the vessel 703 may include estimating one or both of an area or a volume of the vessel 703.

At 860, a food portion is estimated based on the estimated plate size. The portion size may be estimated by the processing component 180. In some embodiments, the processing component 180 may estimate the portion size by determining a percentage of the vessel that is covered by the food in the received image and estimating the size of the portion based on the percentage and the estimated size of the vessel. After estimating the size of the food portion, the processing component 180 may record the determined size and/or may estimate an amount of calories consumed by the wearer of the sensor 100.

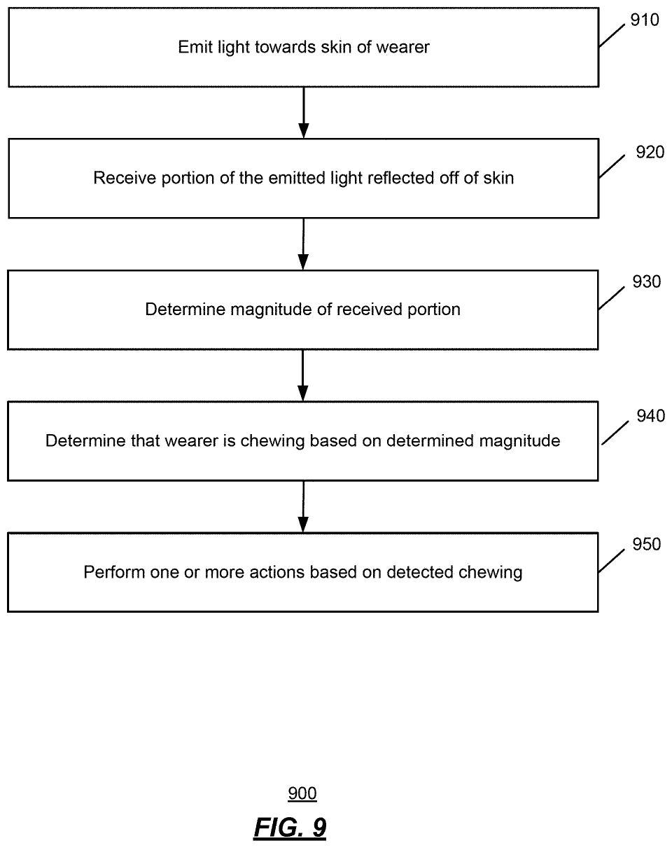

FIG. 9 is an illustration of a method 900 for detecting when a wearer of a sensor 100 is chewing.

At 910, light is emitted towards the skin of a wearer. The light may be emitted by an IR sensor 160 associated with chewing sensor 100. The IR sensor 160 may be part of the chewing sensor 100 and may be attached to the temples of a pair of glasses that are worn by the wearer. The IR sensor 160 may emit infra-red light towards the skin of the wearer, and in particular the skin of the wearer that covers a muscle associated with chewing such as the temporalis muscle.

At 920, a portion of the emitted light is received. A portion of the emitted light that is reflected off of the skin of the wearer is received by IR sensor 160. As may be appreciated, the size or portion of the light that is received changes based on the state of the temporalis muscle.

At 930, a magnitude of the received portion of light is determined. The magnitude of the light may be determined by a sensor circuit 170 associated with the chewing sensor 100. The sensor circuit 170 may receive a portion of light and may output a value that is proportional to the magnitude of the received portion of light.

At 940, that the wearer is chewing is determined based on the determined magnitude. That the wearer is chewing may be determined by the processing component 180 comparing the magnitude to a magnitude that is associated with chewing. In one embodiment, the processing component 180 may compare the changes in magnitude observed over some duration of time. As may be appreciated, as a wearer chews, the determined magnitude may increase and decrease as the temporalis muscle is activated and deactivated. If the patterns in magnitude changes match a pattern associated with chewing the processing component 180 may determine that the wearer is chewing.

At 950, one or more actions are performed based on the determined chewing. The one or more actions may include recording the time when the chewing was determined, counting the total number of chews for a wearer, recording the duration of time associated with the chewing, and estimating the size or portion of the food being eaten based on the number of chews. Other actions may be performed.

FIG. 10 shows an exemplary computing environment in which example embodiments and aspects may be implemented. The computing system environment is only one example of a suitable computing environment and is not intended to suggest any limitation as to the scope of use or functionality.

Numerous other general purpose or special purpose computing system environments or configurations may be used. Examples of well-known computing systems, environments, and/or configurations that may be suitable for use include, but are not limited to, personal computers, servers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 10, an exemplary system for implementing aspects described herein includes a computing device, such as computing device 1000. In its most basic configuration, computing device 1000 typically includes at least one processing unit 1002 and memory 1004. Depending on the exact configuration and type of computing device, memory 1004 may be volatile (such as random access memory (RAM)), non-volatile (such as read-only memory (ROM), flash memory, etc.), or some combination of the two. This most basic configuration is illustrated in FIG. 10 by dashed line 1006.

Computing device 1000 may have additional features/functionality. For example, computing device 1000 may include additional storage (removable and/or non-removable) including, but not limited to, magnetic or optical disks or tape. Such additional storage is illustrated in FIG. 10 by removable storage 1008 and non-removable storage 1010.

Computing device 1000 typically includes a variety of tangible computer readable media. Computer readable media can be any available tangible media that can be accessed by device 1000 and includes both volatile and non-volatile media, removable and non-removable media.

Tangible computer storage media include volatile and non-volatile, and removable and non-removable media implemented in any method or technology for storage of information such as computer readable instructions, data structures, program modules or other data. Memory 1004, removable storage 1008, and non-removable storage 1010 are all examples of computer storage media. Tangible computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 1000. Any such computer storage media may be part of computing device 1000.

Computing device 1000 may contain communications connection(s) 1012 that allow the device to communicate with other devices. Computing device 1000 may also have input device(s) 1014 such as a keyboard, mouse, pen, voice input device, touch input device, etc. Output device(s) 1016 such as a display, speakers, printer, etc. may also be included. All these devices are well known in the art and need not be discussed at length here.

It should be understood that the various techniques described herein may be implemented in connection with hardware or software or, where appropriate, with a combination of both. Thus, the methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computer, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A portion estimation sensor comprising:
a housing;
a processing component contained in the housing;
a distance sensor contained in the housing;
a camera contained in the housing; and
an inertial measurement unit contained in the housing, wherein the processing component is adapted to:
receive an image of a vessel from the camera;
receive data measured by the inertial measurement unit;
based on the data measured by the inertial measurement unit, determine a viewing angle of the camera;
receive a distance measurement from the distance sensor; and
based on the determined viewing angle and the received distance, estimate a size of the vessel in the image.

2. The portion estimation sensor of claim 1, wherein the data measured by the inertial measurement unit comprises heading, pitch, and roll angle.

3. The portion estimation sensor of claim 1, wherein estimating a size of the vessel comprises:
calculating a first plane of an eating surface that includes the vessel;
calculating a height of the vessel;
calculating a second plane that includes a top of the vessel based on the calculated height; and
estimating the size of the vessel based on the first plane, the second plane, the height, and the image.

4. The portion estimation sensor of claim 1, further comprising:
based on the estimated size of the vessel, estimating a volume of food in the vessel.

5. The portion estimation sensor of claim 4, further comprising estimating a type of the food in the vessel.

6. The portion estimation sensor of claim 5, further comprising, based on the estimated type and estimated volume of food, computing an energy and nutrient content of the food in the vessel.

7. The portion estimation sensor of claim 1, wherein the housing is part of an AR or VR headset or goggles.

8. A method comprising:
receiving an image of a vessel from a camera;
receiving data measured by an inertial measurement unit;
based on the data measured by the inertial measurement unit, determining a viewing angle of the camera;
receiving a distance measurement from a distance sensor; and
based on the determined viewing angle and the received distance, estimating a size of the vessel in the image.

9. The method of claim 8, wherein the data measured by the inertial measurement unit comprises heading, pitch, and roll angle.

10. The method of claim 8, wherein estimating a size of the vessel comprises:
calculating a first plane of an eating surface that includes the vessel;
calculating a height of the vessel;
calculating a second plane that includes a top of the vessel based on the calculated height; and
estimating the size of the vessel based on the first plane, the second plane, the height, and the image.

11. The method of claim 8, further comprising:
based on the estimated size of the vessel, estimating a volume of food in the vessel;
estimating a type of the food in the vessel; and
estimating an energy and nutrient content of the food in the vessel based on the estimated volume and the estimated type.

12. The method of claim 8, wherein the camera, inertial measurement unit, and distance sensor are adapted to attach to a pair of glasses.

13. The method of claim 8, wherein the method is performed by an AR or VR headset or goggles.

14. A chewing sensor comprising:
a housing;
a processing component contained in the housing; and
a light sensor contained in the housing, wherein the light sensor is adapted to:
emit light towards the skin of a wearer of the chewing sensor; and
receive a portion of the emitted light reflected from the skin of the wearer of the chewing sensor; and wherein the processing component is adapted to determine if the wearer of the chewing sensor is chewing based on the portion of the emitted light.

15. The chewing sensor of claim 14, wherein the processing component adapted to determine if the wearer of the chewing sensor is chewing based on the portion of the emitted light comprises the processing component adapted to determine if a magnitude of the portion exceeds a threshold or if computed metrics satisfy certain criteria

16. The chewing sensor of claim 14, wherein the chewing sensor is a non-contact sensor.

17. The chewing sensor of claim 14, wherein the light sensor emits light towards a temporalis muscle of the wearer.

18. The chewing sensor of claim 14, wherein the housing is adapted to attach to a pair of glasses.

19. The chewing sensor of claim 14, wherein the housing is part of an AR or VR headset or goggles.

20. The chewing sensor of claim 14, wherein the emitted light is infra-red light.
